(19) United States
(12) Patent Application Publication
(10) Pub. No.: US 2003/0148955 A1
(43) Pub. Date: Aug. 7, 2003

(54) Soluble tumor necrosis factor receptor treatment of medical disorders

(76) Inventor: John D. Pluenneke, Parkville, MO (US)

Correspondence Address: IMMUNEX CORPORATION, LAW DEPARTMENT, 51 UNIVERSITY STREET, SEATTLE, WA, 98101

(21) Appl. No.: 10/314,618

(22) Filed: Dec. 9, 2002

Related U.S. Application Data
(63) Continuation-in-part of application No. 09/778,403, filed on Feb. 7, 2001, now abandoned.
Application No. 09/778,403 is a continuation-in-part of application No. 09/726,781, filed on Nov. 29, 2000.
Application No. 09/726,781 is a continuation-in-part of application No. 09/602,351, filed on Jun. 23, 2000.
Application No. 09/602,351 is a continuation-in-part of application No. PCT/US00/10565, filed on Apr. 19, 2000.
Application No. 09/602,351 is a continuation-in-part of application No. 09/373,828, filed on Aug. 13, 1999, now abandoned.
(60) Provisional application No. 60/184,864, filed on Feb. 25, 2000.
(60) Provisional application No. 60/164,676, filed on Nov. 10, 1999.
(60) Provisional application No. 60/148,234, filed on Aug. 11, 1999.
(60) Provisional application No. 60/143,959, filed on Jul. 15, 1999.
(60) Provisional application No. 60/134,320, filed on May 14, 1999.
(60) Provisional application No. 60/130,074, filed on Apr. 19, 1999.

(51) Int. Cl. A61K 38/17

(52) U.S. Cl. 514/12

(57) ABSTRACT

The invention pertains to methods and compositions for reducing resistance to STI 571 in a chronic myelogenous leukemia patient by administering a TNFα inhibitor, such as recombinant TNFR:Fc to such patient.

This application is a continuation-in-part of U.S. patent application Ser. No. 09/778,403, filed Feb. 7, 2001, which is a continuation-in-part of Ser. No. 09/726,781, filed Nov. 29, 2000, which is a continuation-in-part of Ser. No. 09/602,351, filed Jun. 23, 2000, which is a continuation-in-part of PCT/US00/10565, filed Apr. 19, 2000, (claiming the benefit of priority from U.S. provisional applications 60/184,864, filed Feb. 25, 2000, and 60/164,676, filed Nov. 10, 1999), which is a continuation-in-part of 09/373,828, filed Aug. 13, 1999 (claiming the benefit of priority from U.S. provisional applications 60/148,234, filed Aug. 11, 1999; 60/143,959, filed Jul. 15, 1999; 60/134,320, filed May 14, 1999; and 60/130,074, filed Apr. 19, 1999).

### FIELD OF THE INVENTION

The invention pertains to methods for treating various medical disorders that are characterized by abnormal or excessive TNFα levels by administering a TNFα antagonist, such as a soluble TNFα. The TNFα inhibitor may be administered in combination with other biologically active molecules.

### BACKGROUND OF THE INVENTION

The pleiotropic cytokine tumor necrosis factor alpha (TNFα) is associated with inflammation and binds to cells through membrane receptor molecules, including two molecules having molecular weights of approximately 55 kDa and 75 kDa (p55 and p75). In addition to binding TNFα, the p55 and p75 TNFα receptors mediate the binding to cells of homotrimers of TNFβ, which is another cytokine associated with inflammation and which shares structural similarities with TNFα (e.g., see Cosman, Blood Cell Biochem 7:51-77, 1996). TNFβ is also known as lymphotoxin-α (LTα).

It has been proposed that a systemic or localized excess of TNFα contributes to the progression of numerous medical disorders. For example, patients with chronic heart failure have elevated levels of serum TNFα, which have been shown to increase with disease progression (see, for example, Levine et al., N Eng J Med 323:236-241, 1990). A variety of other diseases are associated with elevated levels of TNFα (see, for example, Feldman et al., Transplantation Proceedings 30:4126-4127, 1998).

It has been suggested that the suppression of TNFα might be beneficial in patients suffering from various disorders characterized by abnormal or excessive TNFα expression. However, although progress has been made in devising effective treatment for such diseases, improved medicaments and methods of treatment are needed.

### SUMMARY OF THE INVENTION

Provided herein are methods for treating a number of medical disorders characterized by abnormal TNFα expression by administering an antagonist of TNFα, such as a soluble TNFα receptor, for a period of time sufficient to induce a sustained improvement in the patient's condition. TNFα inhibitors may be administered in combination with other biologically active molecules.

In one embodiment of the invention, TNFR:Fc is used to prevent or reduce resistance to imatinib in a chronic myelogenous leukemia patient by administering TNFR:Fc to a chronic myelogenous leukemia patient who is being treated concurrently with imatinib. In another embodiment of the invention, TNFR:Fc is used to prevent or reduce resistance to UCN-01 by administering TNFR:Fc to a patient who suffers from gastrointestinal cancer and who is being concurrently treated with UCN-01. For these methods, the TNFR:Fc may be administered one or more times per week, for example, one, two or three times per week. One suitable mode of administration for the TNFR:Fc is by subcutaneous injection. When the patient is an adult, suitable doses for injected TNFR:Fc include 5-12 mg/m² of body surface area, or 25 mg or 50 mg per dose. If the patient is a pediatric patient, the TNFR:Fc may be administered by subcutaneous injection one or more times per week at a dose of 0.4 mg/kg of body weight, up to a maximum of 25 mg per dose. A patient receiving TNFR:Fc for these purposes also may be treated concurrently with an IL-1 inhibitor.

### DETAILED DESCRIPTION OF THE INVENTION

This invention provides compounds, compositions and methods for treating a mammalian patient, including a human patient, who is suffering from a medical disorder that is characterized by abnormal or elevated expression of TNFα. For purposes of this disclosure, the terms “illness,” “disease,” “medical condition,” “abnormal condition” and the like are used interchangeably with the term “medical disorder.”

The subject methods involve administering to the patient a soluble TNFα antagonist that is capable of reducing the effective amount of endogenous biologically active TNFα, such as by reducing the amount of TNFα produced, or by preventing the binding of TNFα to its cell surface receptor (TNFR). Antagonists capable of inhibiting this binding include receptor-binding peptide fragments of TNFα, antisense oligonucleotides or ribozymes that inhibit TNFα production, antibodies directed against TNFα, and recombinant proteins comprising all or portions of receptors for TNFα or modified variants thereof, including genetically-modified muteins, multimeric forms and sustained-release formulations. In other embodiments of the invention, the diseases discussed herein are treated with molecules that inhibit the formation of the IgA-α₁AT complex, such as the peptides disclosed in EP 0 614 464 B, or antibodies against this complex. The hereindescribed conditions also may be treated with the TNFα-inhibiting disaccharides, sulfated derivatives of glucosamine or other similar carbohydrates described in U.S. Pat. No. 6,020,323. In addition, the hereindescribed diseases may be treated with the peptide TNFα inhibitors disclosed in U.S. Pat. No. 5,641,751 and U.S. Pat. No. 5,519,000, and the D-amino acid-containing peptides described in U.S. Pat. No. 5,753,628. In addition, the conditions described herein may be treated with inhibitors of TNFα converting enzyme.

Other compounds suitable for treating the diseases described herein include small molecules such as thalidomide or thalidomide analogs, pentoxifylline, or matrix metalloproteinase (MMP) inhibitors or other small molecules. The foregoing small molecules can be administered concurrently with TNFR:Fc or antibodies against TNFα. Suitable MMP inhibitors for this purpose include, for example, those described in U.S. Pat. Nos. 5,883,131, 5,863,949 and 5,861,510 as well as the mercapto alkyl peptidyl compounds described in U.S. Pat. No. 5,872,146. Other small molecules capable of reducing TNFα production, include, for example, the molecules described in U.S. Pat. Nos. 5,508,300, 5,596,013 and 5,563,143, any of which can be administered in combination with TNFα inhibitors such as soluble TNFRs or antibodies against TNFα. Additional small molecules useful for treating the TNFα-mediated diseases described herein include the MMP inhibitors that are described in U.S. Pat. No. 5,747,514, U.S. Pat. No. 5,691,382, as well as the hydroxamic acid derivatives described in U.S. Pat. No. 5,821,262. The diseases described herein also may be treated with small molecules that inhibit phosphodiesterase IV and TNFα production, such as substituted oxime derivatives (WO 96/00215), quinoline sulfonamides (U.S. Pat. No. 5,834,485), aryl furan derivatives (WO 99/18095) and heterobicyclic derivatives (WO 96/01825; GB 2 291 422 A). Also useful are thiazole derivatives that suppress TNFα and IFNγ (WO 99/15524), as well as xanthine derivatives that suppress TNFα and other proinflammatory cytokines (see, for example, U.S. Pat. No. 5,118,500, U.S. Pat. No. 5,096,906 and U.S. Pat. No. 5,196,430). Additional small molecules useful for treating the hereindescribed conditions include those disclosed in U.S. Pat. No. 5,547,979.

Also included among the TNFα inhibitors of the invention are antisense oligonucleotides that act to directly block the translation of mRNA by hybridizing to targeted mRNA and preventing polypeptide translation. Antisense oligonucleotides are suitable for use in treating any of the medical disorders disclosed herein, either alone or in combination with other TNFα inhibitors, such as TNFR:Fc, or in combination with other agents for treating the same condition. Antisense molecules of the invention may interfere with the translation of TNFα, a TNFα receptor, or an enzyme in the metabolic pathways for the synthesis of TNFα. Absolute complementarity, although preferred, is not required. A sequence “complementary” to a portion of a nucleic acid, as referred to herein, means a sequence having sufficient complementarity to be able to hybridize with the nucleic acid, forming a stable duplex (or triplex, as appropriate). The ability to hybridize will depend on both the degree of complementarity and the length of the antisense nucleic acid. Oligonucleotides that are complementary to the 5′ end of the message, e.g., the 5′ untranslated sequence up to and including the AUG initiation codon, should work most efficiently at inhibiting translation. However, oligonucleotides complementary to either the 5′- or 3′- non- translated, non-coding regions of the targeted transcript can be used. Oligonucleotides complementary to the 5′ untranslated region of the mRNA should include the complement of the AUG start codon.

Antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides. In one embodiment, it will contain 18-21 nucleotides.

The backbone of antisense oligonucleotides may be chemically modified to prolong the half-life of the oligonucleotide in the body. Suitable modifications for this purpose are known in the art, such as those disclosed, for example, in U.S. Pat. No. 6,114,517, which describes the use for this purpose of phosphorothioates, phosphorodithioates, phosphotriesters, aminoalkylphosphotriesters, methyl and other alkyl phosphonates, various phosphonates, phosphinates, and phosphoramidates and so on.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810, published Dec. 15, 1988), or hybridization-triggered cleavage agents or intercalating agents. (See, e.g., Zon, 1988, Pharm. Res. 5:539-549). The antisense molecules should be delivered to cells which express the targeted transcript.

Antisense oligonucleotides can be administered parenterally, including by intravenous or subcutaneous injection, lipofection, or they can be incorporated into formulations suitable for oral administration, such as, for example, ISIS 104838, which targets TNFα. A number of methods have been developed for delivering antisense DNA or RNA to cells; e.g., antisense molecules can be injected directly into the tissue or cell derivation site, or modified antisense molecules, designed to target the desired cells (e.g., antisense linked to peptides or antibodies that specifically bind receptors or antigens expressed on the target cell surface) can be administered systemically. However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous target gene transcripts and thereby prevent translation of the targeted mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Antisense oligonucleotides for suitable for treating diseases associated with elevated TNFα include, for example, the anti-TNFα oligonucleotides described in U.S. Pat. No. 6,080,580, which proposes the use of such oligonucleotides as candidates for testing in animal models of diabetes mellitus, rheumatoid arthritis, contact sensitivity, Crohn's disease, multiple sclerosis, pancreatitis, hepatitis and heart transplant.

Ribozyme molecules designed to catalytically cleave mRNA transcripts can also be used to prevent the translation of mRNAs encoding TNFα, TNFα receptors, or enzymes involved in synthesis of TNFα or TNFRs (see, e.g., PCT WO90/11364; U.S. Pat. No. 5,824,519). Ribozymes useful for this purpose include hammerhead ribozymes (Haseloff and Gerlach, 1988, Nature, 334:585-591), RNA endoribonucleases (hereinafter “Cech-type ribozymes”) such as the one that occurs naturally in Tetrahymena thermophila (known as the IVS, or L-19 IVS RNA) (see, for example, WO 88/04300; Been and Cech, 1986, Cell, 47:207-216). Ribozymes can be composed of modified oligonucleotides (e.g. for improved stability, targeting, etc.) and should be delivered to cells which express the target peptide in vivo. A preferred method of delivery involves using a DNA construct encoding the ribozyme under the control of a strong constitutive pol III or pol II promoter, so that transfected cells will produce sufficient quantities of the ribozyme to destroy endogenous target mRNA, thereby inhibiting its translation.

Alternatively, expression of genes involved in TNFα or TNFR production can be reduced by targeting deoxyribonucleotide sequences complementary to the regulatory region of the target gene (i.e., the target gene promoter and/or enhancers) to form triple helical structures that prevent transcription of the target gene. (see, for example, Helene, 1991, Anticancer Drug Des., 6(6):569-584; Helene, et al., 1992, Ann. N.Y. Acad. Sci., 660:27-36; and Maher, 1992, Bioassays 14(12):807-815).

Anti-sense RNA and DNA, ribozyme, and triple helix molecules of the invention may be prepared by any method known in the art for the synthesis of DNA and RNA molecules, including, for example, solid phase phosphoramidite chemical synthesis. Oligonucleotides can be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al., 1988, Nucl. Acids Res. 16:3209, and methylphosphonate oligonucleotides can be prepared as described by Sarin et al., 1988, Proc. Natl. Acad. Sci. U.S.A. 85:7448-7451. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constituitively or inducibly, depending on the promoter used, can be introduced stably into cell lines.

Endogenous target gene expression can also be reduced by inactivating or “knocking out” the target gene or its promoter using targeted homologous recombination (e.g., see Smithies, et al., 1985, Nature 317:230-234; Thomas and Capecchi, 1987, Cell 51:503-512; Thompson, et al., 1989, Cell 5:313-321). For example, a mutant, non-functional target gene (or a completely unrelated DNA sequence) flanked by DNA homologous to the endogenous target gene (either the coding regions or regulatory regions of the target gene) can be used, with or without a selectable marker and/or a negative selectable marker, to transfect cells that express the target gene in vivo. Insertion of the DNA construct, via targeted homologous recombination, results in inactivation of the target gene. Such approaches are particularly suited in the agricultural field where modifications to ES (embryonic stem) cells can be used to generate animal offspring with an inactive target gene (e.g., see Thomas and Capecchi, 1987 and Thompson, 1989, supra), or in model organisms such as Caenorhabditis elegans where the “RNA interference” (“RNAi”) technique (Grishok A, Tabara H, and Mello CC, 2000, Science 287(5462):2494-2497), or the introduction of transgenes (Dernburg et al., 2000, Genes Dev. 14(13):1578-1583) are used to inhibit the expression of specific target genes. This approach can be adapted for use in humans provided the recombinant DNA constructs are directly administered or targeted to the required site in vivo using appropriate vectors such as viral vectors.

Preferred embodiments of the invention utilize soluble TNFRs as the TNFα antagonist. Soluble forms of TNFRs may include monomers, fusion proteins (also called “chimeric proteins”), dimers, trimers or higher order multimers. In certain embodiments of the invention, the soluble TNFR derivative is one that mimics the 75 kDa TNFR or the 55 kDa TNFR and that binds to TNFα in the patient's body. The soluble TNFR mimics of the present invention may be derived from TNFRs p55 or p75 or fragments thereof. In addition, a soluble TNFR type I or type II for use in the hereindescribed therapies may be conjugated with polyethylene glycol (pegylated) to prolong its serum half-life or to enhance protein delivery. One form of suitable soluble TNFR derived from TNFR p55 is recombinant polyethylene glycol conjugated soluble TNFR p55 (PEG-sTNFR type I), which contains the extracellular domain of TNFR p55. TNFRs other than p55 and p75 also are useful for deriving soluble compounds for treating the various medical disorders described herein, such for example the TNFR that is described in WO 99/04001. Soluble TNFR molecules used to construct TNFR mimics include, for example, analogs or fragments of native TNFRs having at least 20 amino acids, that lack the transmembrane region of the native TNFR, and that are capable of binding TNFα. Antagonists derived from TNFRs compete for TNFα with the receptors on the cell surface, thus inhibiting TNFα from binding to cells, thereby preventing it from manifesting its biological activities. Binding of soluble TNFRs to TNFα or LTα can be assayed using ELISA or any other convenient assay. This invention provides for the use of soluble TNFα receptors in the manufacture of medicaments for the treatment of numerous diseases.

The soluble TNFR polypeptides or fragments of the invention may be fused with a second polypeptide to form a chimeric protein. The second polypeptide may promote the spontaneous formation by the chimeric protein of a dimer, trimer or higher order multimer that is capable of binding a TNFα or a LTα molecule and preventing it from binding to cell-bound receptors. Chimeric proteins used as antagonists include, for example, molecules derived from the constant region of an antibody molecule and the extracellular portion of a TNFR. Such molecules are referred to herein as TNFR-Ig fusion proteins. A preferred TNFR-Ig fusion protein suitable for treating diseases in humans and other mammals is recombinant TNFR:Fc, a term which as used herein refers to “etanercept,” which is a dimer of two molecules of the extracellular portion of the p75 TNFα receptor, each molecule consisting of a 235 amino acid TNFR-derived polypeptide that is fused to a 232 amino acid Fc portion of human IgG₁. Etanercept is currently sold by Immunex Corporation under the trade name ENBREL.® Because the p75 receptor protein that it incorporates binds not only to TNFα, but also to the inflammatory cytokine LTα, etanercept can act as a competitive inhibitor not only of TNFα, but also of LTα. This is in contrast to antibodies directed against TNFα, which cannot inhibit LTα. Also encompassed by the invention are treatments using a compound that comprises the extracellular portion of the 55 kDa TNFR fused to the Fc portion of IgG, including lenercept (Hoffman-La Roche), as well as compositions and combinations containing such a molecule. Additionally, onercept may be used, which is a fusion protein comprising the extracellular portion of p55 and the Fc portion of an immunoglobulin (Serono; CAS registry number 199685-57-9). Encompassed also are therapeutic methods involving the administration of soluble TNFRs derived from the extracellular regions of TNFα receptor molecules other than the p55 and p75 TNFRs, such as for example the TNFR described in WO 99/04001, including TNFR-Ig's derived from this TNFR. Other TNFα inhibitors suitable for use in the subject methods include the fully human anti-TNFα antibody D2E7 (HUMIRA®, known generically as “adalimumab;” Abbott Laboratories) and afelimomab, a murine monoclonal antibody F(ab′)2 fragment that neutralizes human TNFα (SEGARD®; Knoll A G).

In one preferred embodiment of the invention, sustained-release forms of soluble TNFRs are used, including sustained-release forms of TNFR:Fc. Sustained-release forms suitable for use in the disclosed methods include, but are not limited to, TNFRs that are encapsulated in a slowly-dissolving biocompatible polymer (such as the alginate microparticles described in U.S. Pat. No. 6,036,978 or the polyethylene-vinyl acetate and poly(lactic-glucolic acid) compositions described in U.S. Pat. No. 6,083,534), admixed with such a polymer (including topically applied hydrogels), and or encased in a biocompatible semi-permeable implant.

In accord with this invention, medical disorders characterized by abnormal or excess expression of TNFα are administered a therapeutically effective amount of a TNFα inhibitor. The TNFα inhibitor may be a TNFα-binding soluble TNFα receptor, preferably TNFR:Fc. As used herein, the phrase “administering a therapeutically effective amount” of a therapeutic agent means that the patient is treated with the agent in an amount and for a time sufficient to induce a sustained improvement over baseline in at least one indicator that reflects the severity of the disorder. An improvement is considered “sustained” if the patient exhibits the improvement on at least two occasions separated by one or more weeks. The degree of improvement is determined based on signs or symptoms, and determinations may also employ questionnaires that are administered to the patient, such as quality-of-life questionnaires.

Various indicators that reflect the extent of the patient's illness may be assessed for determining whether the amount and time of the treatment is sufficient. The baseline value for the chosen indicator or indicators is established by examination of the patient prior to administration of the first dose of the etanercept or other TNFα inhibitor. Preferably, the baseline examination is done within about 60 days of administering the first dose. If the TNFα antagonist is being administered to treat acute symptoms, such as for example to treat a traumatic knee injury, the first dose is administered as soon as practically possible after the injury has occurred.

Improvement is induced by administering TNFR:Fc or other TNFα antagonist until the patient manifests an improvement over baseline for the chosen indicator or indicators. In treating chronic conditions, this degree of improvement is obtained by repeatedly administering this medicament over a period of at least a month or more, e.g., for one, two, or three months or longer, or indefinitely. A period of one to six weeks, or even a single dose, often is sufficient for treating acute conditions. For injuries or acute conditions, a single dose may be sufficient.

Although the extent of the patient's illness after treatment may appear improved according to one or more indicators, treatment may be continued indefinitely at the same level or at a reduced dose or frequency. Once treatment has been reduced or discontinued, it later may be resumed at the original level if symptoms should reappear.

Any efficacious route of administration may be used to therapeutically administer TNFR:Fc or other TNFα antagonists. If injected, TNFR:Fc can be administered, for example, via intra-articular, intravenous, intramuscular, intralesional, intraperitoneal or subcutaneous routes by bolus injection or by continuous infusion. Other suitable means of administration include sustained release from implants, aerosol inhalation, eyedrops, oral preparations, including pills, syrups, lozenges or chewing gum, and topical preparations such as lotions, gels, sprays, ointments or other suitable techniques. Alternatively, proteinaceous TNFα inhibitors, such as a soluble TNFR, may be administered by implanting cultured cells that express the protein, for example, by implanting cells that express TNFR:Fc. In one embodiment, the patient's own cells are induced to produce TNFR:Fc by transfection in vivo or ex vivo with a DNA that encodes TNFR:Fc. This DNA can be introduced into the patient's cells, for example, by injecting naked DNA or liposome-encapsulated DNA that encodes TNFR:Fc, by infection with a viral vector expressing the DNA, or by other means known in the art. Alternatively, agents may be introduced into a patient's cells that selectively enhance the production of the patient's endogenous secreted soluble TNFR type II. When TNFR:Fc is administered in combination with one or more other biologically active compounds, these may be administered by the same or by different routes, and may be administered simultaneously, separately or sequentially.

TNFR:Fc or other soluble TNFRs or other TNF inhibitors preferably are administered in the form of a physiologically acceptable composition comprising purified recombinant protein in conjunction with physiologically acceptable carriers, excipients or diluents. Such carriers are nontoxic to recipients at the dosages and concentrations employed. Ordinarily, the preparation of such compositions entails combining the TNFα antagonist with buffers, antioxidants such as ascorbic acid, low molecular weight polypeptides (such as those having fewer than 10 amino acids), proteins, amino acids, carbohydrates such as glucose, sucrose or dextrins, chelating agents such as EDTA, glutathione and other stabilizers and excipients. Neutral buffered saline or saline mixed with conspecific serum albumin are exemplary appropriate diluents. In accordance with appropriate industry standards, preservatives may also be added, such as benzyl alcohol. TNFR:Fc preferably is formulated as a lyophilizate using appropriate excipient solutions (e.g., sucrose) as diluents. Suitable components are nontoxic to recipients at the dosages and concentrations employed. Further examples of components that may be employed in pharmaceutical formulations are presented in Remington's Pharmaceutical Sciences, 16^{th} Ed., Mack Publishing Company, Easton, Pa., 1980.

Appropriate dosages can be determined in standard dosing trials, and may vary according to the chosen route of administration. The amount and frequency of administration will depend on such factors as the nature and severity of the indication being treated, the desired response, the age and condition of the patient, and so forth.

In one embodiment of the invention, the TNFα inhibitor is a soluble TNF-Ig, such as TNFR:Fc. TNFR:Fc may administered one time per week to treat the various medical disorders disclosed herein, in another embodiment is administered at least two times per week, and in another embodiment is administered at least three times per week. An adult patient is a person who is 18 years of age or older. If injected, the effective amount of TNFR:Fc per adult dose ranges from 1-20 mg/m² of body surface area, and preferably is about 5-12 mg/m². Alternatively, a flat dose may be administered, whose amount may range from 5-100 mg/dose. Exemplary dose ranges for a flat dose to be administered by subcutaneous injection are 5-25 mg/dose, 25-50 mg/dose and 50-100 mg/dose. In one embodiment of the invention, the various indications described below are treated by administering a preparation acceptable for injection containing TNFR:Fc at 25 mg/dose, or alternatively, containing 50 mg per dose. The 25 mg or 50 mg dose may be administered repeatedly, particularly for chronic conditions. If a route of administration other than injection is used, the dose is appropriately adjusted in accord with standard medical practices. In many instances, an improvement in a patient's condition will be obtained by injecting a dose of about 25 mg of TNFR:Fc one to three times per week over a period of at least three weeks, or a dose of 50 mg of TNFR:Fc one or two times per week for at least three weeks, though treatment for longer periods may be necessary to induce the desired degree of improvement. For incurable chronic conditions, the regimen may be continued indefinitely, with adjustments being made to dose and frequency if such are deemed necessary by the patient's physician.

For pediatric patients (age 4-17), a suitable regimen involves the subcutaneous injection of 0.4 mg/kg of body weight, up to a maximum of 25 mg per dose of TNFR:Fc, administered by subcutaneous injection one or more times per week.

The invention further includes the administration of a soluble TNFR, such as TNFR:Fc, concurrently with one or more other drugs that are administered to the same patient in combination with the soluble TNFR, each drug being administered according to a regimen suitable for that medicament. “Concurrent administration” encompasses simultaneous or sequential treatment with the components of the combination, as well as regimens in which the drugs are alternated, or wherein one component is administered long-term and the other(s) are administered intermittently. Components may be administered in the same or in separate compositions, and by the same or different routes of administration. Examples of drugs to be administered concurrently include but are not limited to antivirals, antibiotics, analgesics, corticosteroids, antagonists of inflammatory cytokines, DMARDs and non-steroidal anti-inflammatories. DMARDs that can be administered in combination with the subject TNFα inhibitors such as TNFR:Fc include azathioprine, cyclophosphamide, cyclosporine, hydroxychloroquine sulfate, methotrexate, leflunomide, minocycline, penicillarmine, sulfasalazine and gold compounds such as oral gold, gold sodium thiomalate and aurothioglucose. Additionally, TNFR:Fc may be combined with a second TNFα antagonist, including an antibody against TNFα or TNFR, a TNFα-derived peptide that acts as a competitive inhibitor of TNFα (such as those described in U.S. Pat. No. 5,795,859 or U.S. Pat. No. 6,107,273), a TNFR-IgG fusion protein other than etanercept, such as one containing the extracellular portion of the p55 TNFα receptor, a soluble TNFR other than an IgG fusion protein, or other molecules that reduce endogenous TNFα levels, such as inhibitors of the TNFα converting enzyme (see e.g., U.S. Pat. No. 5,594,106), or any of the small molecules or TNFα inhibitors that are described above, including pentoxifylline or thalidomide or derivatives thereof.

Thalidomide or thalidomide derivatives may be administered concurrently with a TNF inhibitor to treat, for example, hematologic and oncologic disorders. Examples of such disorders, any of which may be treated with a TNF inhibitor alone, include graft-versus-host disease, myelodysplastic syndromes, aplastic anemia, sickle cell vasocclusive crisis, acute myelogenous leukemia (AML), chronic lymphocytic leukemia (CLL), hairy cell leukemia, paraneoplastic syndrome of cachexia and hypercalcemia, multiple myeloma and POEMS syndrome (polyneuropathy, organomegaly, endocrinopathy, monoclonal gammopathy, and skin changes), myelofibrosis/myeloid metaplasia, Kaposi's sarcoma, cachexia associated with cancer, amyloidosis, anemia of chronic disease, squamous cell carcinoma, idiopathic thrombocytopenic purpura (ITP), thrombotic thrombocytopenic purpura (TTP), autoimmune hemolytic anemia and beta thalassemia. In one embodiment of the invention, the TNF inhibitor used in combination with thalidomide is TNFR:Fc.

If an antibody against TNFα is used as the TNFα inhibitor, a preferred dose range is 0.1 to 20 mg/kg, and more preferably is 1-10 mg/kg. Another preferred dose range for anti-TNFα antibody is 0.75 to 7.5 mg/kg of body weight. Humanized antibodies are useful as therapeutic agents, that is, antibodies in which only the antigen-binding portion of the antibody molecule is derived from a non-human source. An exemplary humanized antibody for treating the hereindescribed diseases is infliximab (sold by Centocor as REMICADE®), which is a chimeric IgG1κ monoclonal antibody having an approximate molecular weight of 149,100 daltons. Infliximab is composed of human constant and murine variable regions, and binds specifically to human TNFα. Other suitable anti-TNFα antibodies include the humanized antibodies D2E7 (adalimumab), afelimomab (Knoll) and CDP571 (Celltech Therapeutics), CDP870, which is a pegylated humanized monoclonal antibody to TNFα (Celltech Therapeutics and Pharmacia) and the antibodies described in EP 0 516 785 B1, U.S. Pat. No. 5,656,272, EP 0492448 A1. Modes of administration for such antibodies include subcutaneous injection or intravenous administration.

In one preferred embodiment of the invention, the various medical disorders disclosed herein as being treatable with inhibitors of TNFcc are treated in combination with another cytokine or cytokine inhibitor. For example, a soluble TNFR such as TNFR:Fc may be administered in a composition that also contains a compound that inhibits the interaction of other inflammatory cytokines with their receptors. Examples of cytokine inhibitors used in combination with TNFR:Fc include, for example, antagonists of TGFβ, Il-6 or Il-8. TNFα inhibitors such as TNFR:Fc also may be administered in combination with the cytokines GM-CSF, IL-2 and inhibitors of protein kinase A type 1 to enhance T cell proliferation in HIV-infected patients who are receiving anti-retroviral therapy. In addition, TNFα inhibitors may be combined with inhibitors of IL-13 to treat Hodgkin's disease.

Nerve growth factors also can be combined with TNFα inhibitors to treat certain conditions. Such conditions include neurodegenerative diseases, spinal cord injury and multiple sclerosis. Other conditions treatable with this combination are glaucoma and diabetes.

In addition, the subject invention provides methods for treating a human patient in need thereof, the method involving administering to the patient a therapeutically effective amount of a TNFα inhibitor and an IL-4 inhibitor. IL-4 can induce an inflammatory effect in some instances, such as in asthma, a condition in which over-expression of IL-4 in the lungs causes epithelial cell hypertrophy and an accumulation of lymphocytes, eosinophils and neutrophils. This response is representative of the main features of the proinflammatory response induced by other Th2 cytokines. TNFα induces the proliferation of activated T cells and also plays a role in many diseases where IL-4 has a proinflammatory effect. In such diseases, the infiltration and proliferation of Th2 cells is fueled by TNFα, which cells in turn overproduce IL-4. In such settings, the suppression of both IL-4 and TNFα will have a greater impact on the disease than treatment that suppresses only one of these cytokines.

Combinations of TNFα inhibitors and IL-4 inhibitors preferably are administered one or more times per week. A preferred mode of administration is subcutaneous injection. Suitable dose ranges for IL-4 antagonists include doses of from about 1 ng/kg/day to about 10 mg/kg/day, more preferably from about 500 ng/kg/day to about 5 mg/kg/day, and most preferably from about 5 μg/kg/day to about 2 mg/kg/day, administered to adults one time per week, two times per week, or three or more times per week. If injected, suitable doses may range from 1-20 mg/m², and preferably is about 5-12 mg/m². Alternatively, a flat dose of about 5-100 mg/dose may be used, preferably about 20-30 mg per dose. For pediatric patients (age 4-17), one suitable regimen involves subcutaneous injection of 0.4 mg/kg, up to a maximum dose of 25 mg of IL-4R, administered two or three times per week. Another embodiment is directed to aerosol pulmonary administration, for example by nebulizer, which optimally will deliver a dose of 3 or more mg of a soluble IL-4R, and is taken at least once a week. Aeresolized IL-4R may be administered orally or nasally. One illustrative embodiment involves subcutaneous injection of a soluble human IL-4R once a week, at a dose of 1.5 to 3 mg. Doses will be adjusted as needed by the patient's physician in accord with standard medical practices.

Conditions effectively treated by a combination of a TNFα inhibitor and an IL-4 inhibitor include conditions in which a Th2-type immune response plays a role or conditions in which IL-4 plays a role in the inflammatory response. Lung disorders in which IL-4 plays a role include asthma, chronic obstructive pulmonary disease, pulmonary alveolar proteinosis, bleomycin-induced pneumopathy and fibrosis, radiation-induced pulmonary fibrosis, cystic fibrosis, collagen accumulation in the lungs, and ARDS, all of which may be treated with combinations of a TNFα inhibitor and an IL-4 inhibitor. Combinations of TNFα inhibitors and IL-4 inhibitors also are useful for treating patients suffering from various skin disorders, including but not limited to dermatitis herpetiformis (Duhring's disease), atopic dermatitis, contact dermatitis, urticaria (including chronic idiopathic urticaria), and autoimmune blistering diseases, including pemphigus vulgaris and bullous pemphigoid. Other diseases treatable with the combination of a TNFα inhibitor and an IL-4 inhibitor include myasthenia gravis, sarcoidosis, including pulmonary sarcoidosis, scleroderma, reactive arthritis, hyper IgE syndrome, multiple sclerosis and idiopathic hypereosinophil syndrome. The combination is used also for treating allergic reactions to medication and as an adjuvant to allergy immunotherapy.

IL-4 antagonists that may be employed in accordance with the present invention include, but are not limited to, IL-4 receptors (IL-4R) and other IL-4-binding molecules, IL-4 muteins and antibodies that bind specifically with IL-4 or IL-4 receptors thereby blocking signal transduction, as well as antisense oligonucleotides and ribozymes targeted to IL-4 or IL-4R. Antibodies specific for IL-4 or IL-4 receptor may be prepared using standard procedures. Among the IL-4 receptors suitable for use as described herein are soluble fragments of human IL-4R that retain the ability to bind IL-4. Such fragments are capable of binding IL-4, and retain all or part of the IL-4R extracellular region.

After binding to an IL-4 antagonist according to the invention, endogenous IL-4 or IL-4R is thereby hindered or prevented from binding its natural receptor on cell surfaces in vivo, and thus IL-4-mediated biological activities are inhibited. IL-4 antagonists useful for the hereindescribed methods of treatment include molecules that selectively block the synthesis of endogenous IL-4 or IL-4R. IL-4 receptors are described in U.S. Pat. No. 5,599,905; Idzerda et al., J. Exp. Med. 171:861-873 (1990) (human IL-4R); and Mosley et al., Cell 59:335-348 (1989) (murine IL-4R), each of which is hereby incorporated by reference in its entirety. The protein described in those three references is sometimes referred to in the scientific literature as IL-4Rα. Unless otherwise specified, the terms “IL-4R” and “IL-4 receptor” as used herein encompass this protein in various forms that are capable of functioning as IL-4 antagonists, including but not limited to soluble fragments, fusion proteins, oligomers, and variants that are capable of binding IL-4, as described in more detail below. Suitable IL-4Rs include variants in which valine replaces isoleucine at position 50 (see Idzerda et al., 1990), and include slow-release formulations, and PEGylated derivatives (modified with polyethylene glycol) are contemplated, as well as recombinant fusion proteins comprising heterologous polypeptides fused to the N-terminus or C-terminus of an IL-4R polypeptide, including signal peptides, immunoglobulin Fc regions, poly-His tags or the FLAG® polypeptide described in Hopp et al., Bio/Technology 6:1204, 1988, and U.S. Pat. No. 5,011,912, as well as fusions of IL-4 receptors with oligomer-promoting leucine zipper moieties. Soluble recombinant fusion proteins comprising an IL-4R and immunoglobulin constant regions are described, for example, in EP 464,533.

Various IL-4 antagonists that may be used for the hereindescribed methods of treatment can be identified, for example, by their ability to inhibit ³H-thymidine incorporation in cells that normally proliferate in response to IL-4, or by their ability to inhibit binding of IL-4 to cells that express IL-4R. In one assay for detecting IL-4 antagonists, one measures the ability of a putative antagonist to block the IL-4-induced enhancement of the expression of CD23 on the surfaces of human B cells. For example, B cells isolated from human peripheral blood are incubated in microtiter wells in the presence of IL-4 and the putative antagonist. Following the incubation, washed cells are then incubated with labelled monoclonal antibody against CD23 (available from Pharmingen) to determine the level of CD23 expression. An anti-huIL-4R murine mAb (R&D Systems), previously shown to block the binding and function of both hIL-4 and hIL-13, may used as a positive control for neutralization of CD23 induction by IL-4. Alternatively, suitable IL-4 antagonists may be identified by determining their ability to prevent or reduce the impaired the barrier function of epithelium that results when IL-4 is incubated with the epithelium. For this purpose, one may use confluent monolayers of human epithelial cell lines such as Calu-3 (lung) or T84 (intestinal epithelium). Incubation of such monolayers with IL-4 causes significant damage to their barrier function within about 48 hours. To assay IL-4 antagonists, monolayers may be tested for their permeability, for example, by adding radiolabeled mannitol to cells incubated with IL-4 in the presence or absence of an antagonist. Alternatively, transepithelial resistance (indicating an intact barrier) may be determined using a voltmeter.

The present invention also relates to the use of the disclosed TNFα inhibitors, such as TNFR:Fc, in the manufacture of a medicament for the prevention or therapeutic treatment of each medical disorder disclosed herein.

The disclosed TNFα inhibitors, compositions and combination therapies described herein are useful in medicines for treating bacterial, viral or protozoal infections, and complications resulting therefrom. One such disease is Mycoplasma pneumonia. In addition, provided herein is the use of TNFR:Fc to treat AIDS and related conditions, such as AIDS dementia complex, AIDS associated wasting, lipidistrophy due to antiretroviral therapy; and Kaposi's sarcoma. Provided herein is the use of TNFR:Fc for treating protozoal diseases, including malaria (including cerebral malaria) and schistosomiasis. Additionally provided is the use of TNFR:Fc to treat erythema nodosum leprosum; bacterial or viral meningitis; tuberculosis, including pulmonary tuberculosis; and pneumonitis secondary to a bacterial or viral infection. Provided also herein is the use of TNFR:Fc to prepare medicaments for treating louse-borne relapsing fevers, such as that caused by Borrelia recurrentis. TNFR:Fc can also be used to prepare a medicament for treating conditions caused by Herpes viruses, such as herpetic stromal keratitis, corneal lesions, and virus-induced corneal disorders. In addition, TNFR:Fc can be used in treating human papillomavirus infections, as well as in treating infectious mononucleosis. TNFR:Fc is used also to prepare medicaments to treat influenza, as well as to treat critical illness polyneuropathy and myopathy (CIPNM), an inflammatory syndrome that occasionally occurs in conjunction with prolonged septic illnesses. The subject TNFα inhibitors are used also to treat transmissible spongiform encephalopathies, which is believed to be mediated by prions.

Another disorder that can be treated with any of the disclosed TNFα inhibitors, pharmaceutical compositions or combination therapies is tropical spastic paraparesis/HTLV-1 associated myelopathy (TSP/HAM). This disease is caused by infection with the human retrovirus HTLV-1. Recent studies have suggested that TNFα may play a role in the decreased glutamate uptake exhibited by HTLV-infected cells (Szymocha et al., J Virol 74:6433-41 (2000)). TSP/HAM is a slowly progressing condition of the spinal cord that causes weakness and muscle stiffness in the legs, often accompanied by a loss of sensation in the feet. Known treatments for this condition include corticosteroids and plasmapheresis. TSP/HAM may be treated with any of the TNFα inhibitors disclosed herein, any of which may be administered concurrently with a corticosteroid, plasmapheresis or both. An exemplary TNFα inhibitor for treating TSP/HAM is TNFR:Fc. Sufficiency of treatment is determined by monitoring the patient for improvement in leg strength, or an arrest of the patient's deterioration or by any other means deemed appropriate by the patient's physician.

Cardiovascular disorders are treatable with the disclosed TNFα inhibitors, pharmaceutical compositions or combination therapies. Examples of cardiovascular disorders treatable with a TNFα antagonist, such as TNFR:Fc, include: aortic aneurisms; arteritis; vascular occlusion, including cerebral artery occlusion; complications of coronary by-pass surgery; ischemia/reperfusion injury; heart disease, including atherosclerotic heart disease, myocarditis, including chronic autoimmune myocarditis and viral myocarditis; heart failure, including chronic heart failure (CHF), cachexia of heart failure; myocardial infarction; restenosis after heart surgery; silent myocardial ischemia; post-implantation complications of left ventricular assist devices; Raynaud's phenomena; thrombophlebitis; vasculitis, including Kawasaki's vasculitis; giant cell arteritis, Wegener's granulomatosis; and Schoenlein-Henoch purpura.

In addition, TNFR:Fc or the other TNF inhibitors disclosed herein may be used in combination with myeloid or angiogenic stem cell therapies for the treatment of cardiovascular disease, including cardiomyopathy of ischemic or non-ischemic origin, post-myocardial infarction angiogenic therapy or treatment for peripheral arterial disease. Stem cells useful for this purpose include mesenchymal stem cells and endothelial precursor cells, such as those found in spleen, fetal liver, bone marrow or circulating blood (U.S. Pat. No. 5,486,359; Deisher T, Drugs 3(6):649-53 (2000); Huss R, Stem Cells 18:1-9 (2000); Huss et al., Stem Cells 18:252-60 (2000)). The TNF antagonists may be given concurrently with stem cell transplants as well as treatments with proliferative or differentiative stem cell growth factors.

TNFα and IL-8 have been implicated as chemotactic factors in athersclerotic abdominal aortic aneurism (Szekanecz et al., Pathobiol 62:134-139 (1994)). Abdominal aortic aneurism may be treated in human patients by administering a soluble TNFR, such as TNFR:Fc, which may be administered in combination with an inhibitor of IL-8, such treatment having the effect of reducing the pathological neovascularization associated with this condition.

Studies have shown that metalloproteinases (MMPs) are a key element in myocardial remodeling and fibrosis. Thus, inhibiting TNFα and the inflammatory response in conjunction with direct inhibition of MMPs will reduce, prevent or reverse disorders such as left ventricular pump dysfunction. This is accomplished by co-administering a TNFα antagonist, such as TNFR:Fc or other antagonist, together with a MMP inhibitor. Alternatively, treatment of left ventricular pump dysfunction may involve administering a TNFα antagonist without the concurrent use of a MMP inhibitor.

A combination of a TNFα inhibitor and one or more other anti-angiogenesis factors may be used to treat solid tumors, thereby reducing the vascularization that nourishes the tumor tissue. Suitable anti-angiogenic factors for such combination therapies include IL-8 inhibitors, angiostatin, endostatin, kringle 5, inhibitors of vascular endothelial growth factor (VEGF), angiopoietin-2 or other antagonists of angiopoietin-1, antagonists of platelet-activating factor and antagonists of basic fibroblast growth factor. Antibodies against vascular endothelial growth factor, such as the recombinant humanized anti-VEGF (AVASTIN™, known generically as “bevacizumab;” Genentech, Inc.), are useful for combination treatments with TNFα inhibitors such as TNFR:Fc.

In addition, the subject TNFα inhibitors, compositions and combination therapies are used to treat chronic pain conditions, such as chronic pelvic pain, including chronic prostatitis/pelvic pain syndrome. As a further example, TNFR:Fc and the compositions and combination therapies of the invention are used to treat post-herpetic pain.

Provided also are methods for using TNFα inhibitors, compositions or combination therapies to treat various disorders of the endocrine system. For example, the TNFα inhibitors are used to treat juvenile onset diabetes (includes autoimmune and insulin-dependent types of diabetes) and also to treat maturity onset diabetes (includes non-insulin dependent and obesity-mediated diabetes). In addition, the subject compounds, compositions and combination therapies are used to treat secondary conditions associated with diabetes, such as diabetic retinopathy, kidney transplant rejection in diabetic patients, obesity-mediated insulin resistance, and renal failure, which itself may be associated with proteinurea and hypertension. Other endocrine disorders also are treatable with these compounds, compositions or combination therapies, including polycystic ovarian disease, X-linked adrenoleukodystrophy, hypothyroidism and thyroiditis, including Hashimoto's thyroiditis (i.e., autoimmune thyroiditis).

Conditions of the gastrointestinal system also are treatable with TNFα inhibitors, compositions or combination therapies, including coeliac disease. In addition, the compounds, compositions and combination therapies of the invention are used to treat Crohn's disease; nausea associated with gastrointestinal disorders or other systemic disorders; ulcerative colitis; idiopathic gastroparesis; cholelithiasis (gallstones); pancreatitis, including chronic pancreatitis and lung injury associated with acute pancreatitis; and ulcers, including gastric and duodenal ulcers.

Included also are methods for using the subject TNFα inhibitors, compositions or combination therapies for treating disorders of the genitourinary system, such as glomerulonephritis, including autoimmune glomerulonephritis, glomerulonephritis due to exposure to toxins or glomerulonephritis secondary to infections with haemolytic streptococci or other infectious agents. Also treatable with the compounds, compositions and combination therapies of the invention are uremic syndrome and its clinical complications (for example, renal failure, anemia, and hypertrophic cardiomyopathy), including uremic syndrome associated with exposure to environmental toxins, drugs or other causes. Further conditions treatable with the compounds, compositions and combination therapies of the invention are complications of hemodialysis; prostate conditions, including benign prostatic hypertrophy, nonbacterial prostatitis and chronic prostatitis; and complications of hemodialysis.

Also provided herein are methods for using TNFα inhibitors, compositions or combination therapies to treat various hematologic and oncologic disorders. For example, TNFR:Fc is used to treat various forms of cancer, including acute myelogenous leukemia, Epstein-Barr virus-positive nasopharyngeal carcinoma, gall bladder carcinoma, glioma, colon, stomach, prostate, renal cell, cervical and ovarian cancers, lung cancer (SCLC and NSCLC), including cancer-associated nausea, cancer-associated cachexia, fatigue, asthenia, paraneoplastic syndrome of cachexia and hypercalcemia. Additional diseases treatable with the subject TNFα inhibitors, compositions or combination therapies are solid tumors, including sarcoma, osteosarcoma, and carcinoma, such as adenocarcinoma (for example, breast cancer) and squamous cell carcinoma. In addition, the subject compounds, compositions or combination therapies are useful for treating leukemia, including chronic or acute myelogenous leukemia, chronic or acute lymphoblastic leukemia and hairy cell leukemia. Other malignancies with invasive metastatic potential can be treated with the subject compounds, compositions and combination therapies, including multiple myeloma. When TNFα inhibitors are used to treat a tumor, this treatment may be administered in combination with antibodies targeted to membrane proteins that are expressed at a high level on the particular tumor being treated. For example, tumors such as breast, ovarian and prostate carcinomas or other Her2-positive tumors, can be administered with TNFR:Fc or other TNFα inhibitors in combination with antibodies against Her2/neu, such as HERCEPTIN® (know generically as “trastuzumab;” Genentech, Inc.). Cancer, for example ovarian cancer or prostate cancer, can be treated by concurrent administration of a TNFα inhibitor, such as TNFR:Fc, and interferon-γ (Windbichler et al., British J Cancer 82(6): 1138-44 (2000)).

In one embodiment of the invention, the TNF inhibitor, such as TNFR:Fc, is administered to cancer patients in combination with a proteasome inhibitor, including patients suffering from hematologic cancers or solid tumors. The proteasome controls the stability of various proteins involved in the cell cycle and apoptosis, such as cyclins and NF-κB (see, for example, Schenkein, Clin Lymphoma 3:49-55 (2002) and Adams, Curr Opin Oncol 14:628-34 (2002)). Proteasome inhibitors can induce apoptosis, thus can sensitize cancer cells to other anti-cancer agents. Exemplary proteasome inhibitors for the subject combinations include, for example, carbobenzoxy-L-leucyl-L-leucyl-Lleucinal (MG132), clasto-lactacystin beta-lactone (A. G. Scientific, Inc.), carbobenzoxy-isoleucyl-(gamma)-t-butyl-L-glutamyl-L-alanyl-L-leucinal (PSI), N-acetyl-leu-leunorleucinal (ALLN), MLN519 (Millennium Pharmaceuticals), [(1R)-3-methyl-1-[[(2S)-3-phenyl-2-[(pyrazinylcarbonyl)amino]propanoyl]amino] [symbol] butyl]boronic acid (PS-341, known generically as “bortezomib;” trade name VELCADE®; Millennium Pharmaceuticals), and carbobenzoxy-L-leucyl-L-leucyl-L-norvalinal (MG 115; Affiniti Research Products). For example, multiple myeloma, ovarian cancer, prostate cancer, breast cancer, hematologic malignancies, such as lymphoma or leukemia, or other tumors may be treated concurrently with a proteasome inhibitor, such as PS-341, and an TNFα inhibitor, such as TNFR:Fc or an antibody against TNFα.

In addition, the disclosed TNFα inhibitors, compositions and combination therapies can be used to treat anemias and hematologic disorders, including anemia of chronic disease, aplastic anemia, including Fanconi's aplastic anemia; idiopathic thrombocytopenic purpura (ITP); myelodysplastic syndromes (including refractory anemia, refractory anemia with ringed sideroblasts, refractory anemia with excess blasts, refractory anemia with excess blasts in transformation); myelofibrosis/myeloid metaplasia; and sickle cell vasocclusive crisis. In addition, TNFα inhibitors, such as TNFR:Fc, are useful for treating chronic idiopathic neutropenia.

Undesired side effects of certain therapies can be treated with TNFα antagonists, such as TNFR:Fc. Such side effects in some instances are mediated by elevated TNFα levels, thus such patients will benefit from treatment with an agent that reduces levels of TNFα. For example, TNFα antagonists may be administered to help combat the nausea associated with chemotherapy or other drug-induced nausea. In addition, TNFα antagonists are used to treat the radiation-induced brain damage associated with radiation treatment for brain tumors. Furthermore, TNF(X antagonists are used to treat the toxicity associated with the administration of monoclonal antibodies directed against antigens present on the surface of particular kinds of cancer cells. For example, the TNFα inhibitors disclosed herein may be used to treat toxicity associated with infusion of CAMPATH 1-H® (known generically as “alemtuzumab;” Berlex Laboratories; see also EP 0328404A1), which is used to treat chronic lymphocytic leukemia. CAMPATH 1-H is a humanized antibody specific for CD52, a cell surface antigen found on monocytes, B cells and T cells. In another embodiment of the invention, the disclosed TNFα inhibitors, such as TNFR:Fc or an antibody against TNFα, may be administered to ameliorate the autoimmune response disorder related to long-term interferon treatment.

In addition, TNFα inhibitors can be used to prevent development of or alleviate drug resistance to agents that are bound by alpha-1-acid glycoprotein (AGP), a protein that capable of binding to small molecules and that preferentially binds to basic molecules. AGP is an acute phase protein that becomes increased in a variety of pathologic conditions, including chronic inflammation, myocardial infarction and cancer. STI 571 (Glivec®, generically known as “imatinib;” Novartis) is an active inhibitor of Bcr-Abl and C-kit kinase activity, and is useful for treating chronic myelogenous leukemia (CML). A mouse model study of CML has shown that AGP binds and inactivates imatinib, thus resulting in a resistance to this drug (Gambacorti-Passerini et al., J Natl Can Inst 92:1641-50 (2000)). The level of AGP in a patient can be lowered by administering pentoxifylline (Voisin et al., Am j Physiol 275:R1412-R1419 (1998)). The subject invention provides methods of preventing or reducing resistance to imatinib by concurrently administering a TNFα inhibitor, such as TNFR:Fc or an antibody against TNFα, to a CML patient who is undergoing treatment with imatinib. UCN-01 (7-hydroxystaurosporine), an agent used to treat gastrointestinal and other solid tumors, also has a propensity for binding to AGP (Senderowicz et al., J Natl Cancer Inst 92(5):376-87 (2000); Noriaki et al., Biol Pharmac Bull 23(7):893-95 (2000); Fuse et al., Cancer Res 59(5):1054-60 (1999); Tamura et al., Proc Annu Meet Am Soc Clin Oncol 18:A611 (1999). Provided herein is a method for preventing or reducing UCN-01 binding to AGP by administering a TNFα inhibitor, such as TNFR:Fc, to a gastrointestinal cancer patient who is being treated concurrently with UCN-01, thereby enhancing the effectiveness of the UCN-01 treatment. Alternatively, patients receiving imatinib or UCN-01 can be treated by the concurrent administration of an IL-1 inhibitor or an TNFα inhibitor together with an IL-1 inhibitor, such as one of the IL-1 inhibitors described in WO 01/87328, which is hereby incorporated by reference in its entirety.

Various lymphoproliferative disorders also are treatable with the disclosed TNFα inhibitors, compositions or combination therapies. These include, but are not limited to autoimmune lymphoproliferative syndrome (ALPS), chronic lymphoblastic leukemia, hairy cell leukemia, chronic lymphatic leukemia, peripheral T-cell lymphoma, small lymphocytic lymphoma, mantle cell lymphoma, follicular lymphoma, Burkitt's lymphoma, Epstein-Barr virus-positive T cell lymphoma, histiocytic lymphoma, Hodgkin's disease, diffuse aggressive lymphoma, acute lymphatic leukemias, T gamma lymphoproliferative disease, cutaneous B cell lymphoma, cutaneous T cell lymphoma (i.e., mycosis fungoides) and Sézary syndrome.

In addition, the subject TNFα inhibitors, compositions and combination therapies are used to treat hereditary conditions such as Gaucher's disease, Huntington's disease, linear IgA disease, and muscular dystrophy.

Other conditions treatable by the disclosed TNFα inhibitors, compositions and combination therapies include those resulting from injuries to the head or spinal cord, and including subdural hematoma due to trauma to the head.

In addition, the disclosed TNFα inhibitors, compositions and combination therapies are used to treat various disorders that involve hearing loss and that are associated with abnormal TNFα expression. One of these is inner ear or cochlear nerve-associated hearing loss that is thought to result from an autoimmune process, i.e., autoimmune hearing loss. This condition currently is treated with steroids, methotrexate and/or cyclophosphamide, which may be administered concurrently with the TNFR:Fc or other TNFα inhibitor. Also treatable with the disclosed TNFα inhibitors, compositions and combination therapies is cholesteatoma, a middle ear disorder often associated with hearing loss.

In addition, the subject invention provides TNFα inhibitors, compositions and combination therapies for the treatment of non-arthritic medical conditions of the bones and joints. This encompasses osteoclast disorders that lead to bone loss, such as but not limited to osteoporosis, including post-menopausal osteoporosis, periodontitis resulting in tooth loosening or loss, and prosthesis loosening after joint replacement (generally associated with an inflammatory response to wear debris). This latter condition also is called “orthopedic implant osteolysis.” Other conditions treatable by administering TNFRα inhibitors, such as TNFR:Fc, include temporal mandibular joint dysfunction (TMJ) and bone loss due to the hypercalcemia of cancer, including metastases to bone, such as, for example, may occur in melanoma or carcinoma of lung, breast, lung, squamous cell carcinoma, head and neck cancer, renal cancer, or prostate cancer.

Other treatments for the hereindescribed diseases include administering TNFR:Fc concurrently with compounds that block the binding of RANK and RANK-ligand, such as antagonistic antibodies against RANK or RANK-ligand, osteoprotegerin or soluble forms of RANK, including RANK:Fc, and soluble forms of RANK-ligand that do not trigger RANK. In one embodiment of the invention, antibodies that specifically bind human RANKL are administered concurrently with a soluble TNFα receptor, such as TNFR:Fc. Soluble forms of RANK suitable for these combinations are described, for example, in U.S. Pat. No. 6,017,729. The concurrent administration of TNFR:Fc together with RANK:Fc or osteoprotegerin is useful for preventing bone destruction in various settings including but not limited to osteoporosis, multiple myeloma or other malignancies that cause bone degeneration, or anti-tumor therapy aimed at preventing metastasis to bone, or bone destruction associated with prosthesis wear debris or with periodontitis. Tumors that are treatable with a combination of a TNFα inhibitor and a RANK inhibitor include breast cancer, lung cancer, melanoma, bone cancer, squamous cell carcinoma, head and neck cancer, renal cancer, prostate cancer and cancers associated with hypercalcemia.

A number of pulmonary disorders also can be treated with the disclosed TNFα inhibitors, compositions and combination therapies. One such condition is adult respiratory distress syndrome (ARDS), which is associated with elevated TNFα, and may be triggered by a variety of causes, including exposure to toxic chemicals, pancreatitis, trauma or other causes. The disclosed compounds, compositions and combination therapies of the invention also are useful for treating broncho-pulmonary dysplasia (BPD); lymphangioleiomyomatosis; pulmonary hypertension; chronic fibrotic lung disease of preterm infants; and idiopathic bronchiectasis. Idiopathic bronchiectasis is a disease in which neutrophils mediate degradation of the proteoglycan component of the bronchial matrix. Proinflammatory mediators in the bronchial secretions of bronchiectasis patients, particularly TNFα, are suspected of enhancing the degradative action of these neutrophils (Shum et al., Am J Respir Crit Care Med 162:1925-31 (2000)). The present invention provides treatment for idiopathic bronchiectasis that comprises administering a TNFα inhibitor, such as TNFR:Fc or another of the TNFα inhibitors described herein. In addition, the compounds, compositions and combination therapies of the invention are used to treat occupational lung diseases, including asbestosis, coal worker's pneumoconiosis, silicosis or similar conditions associated with long-term exposure to fine particles. In other aspects of the invention, the disclosed compounds, compositions and combination therapies are used to treat pulmonary disorders, including chronic obstructive pulmonary disease (COPD) associated with chronic bronchitis or emphysema; fibrotic lung diseases, such as cystic fibrosis, idiopathic pulmonary fibrosis and radiation-induced pulmonary fibrosis; sarcoidosis, including pulmonary sarcoidosis; and allergies, including allergic rhinitis, contact dermatitis, atopic dermatitis and asthma.

Cystic fibrosis is an inherited condition characterized primarily by the accumulation of thick mucus, predisposing the patient to chronic lung infections and obstruction of the pancreas, which results in malabsorption of nutrients and malnutrition. TNFR:Fc may be administered to treat cystic fibrosis. If desired, treatment with TNFR:Fc may be administered concurrently with corticosteroids, mucus-thinning agents such as inhaled recombinant deoxyribonuclease I (such as PULMOZYME®; Genentech, Inc.) or inhaled tobramycin (TOBI®; Pathogenesis, Inc.). TNFR:Fc also may be administered concurrently with corrective gene therapy, drugs that stimulate cystic fibrosis cells to secrete chloride or other yet-to-be-discovered treatments. Sufficiency of treatment may be assessed, for example, by observing a decrease in the number of pathogenic organisms in sputum or lung lavage (such as Haemophilus influenzae, Stapholococcus aureus, and Pseudomonas aeruginosa), by monitoring the patient for weight gain, by detecting an increase in lung capacity or by any other convenient means.

The disclosed TNFα inhibitors, such as TNFR:Fc, compositions and combination therapies are further used to treat conditions of the liver such as hepatitis, including acute alcoholic hepatitis, acute drug-induced or viral hepatitis, hepatitis A, B and C, sclerosing cholangitis, autoimmune hepatitis, idiopathic portal hypertension and inflammation of the liver due to unknown causes. The foregoing liver diseases may be treated with a TNFα inhibitor, such as TNFR:Fc, concurrently with other medications used to treat the same conditions. As an example, TNFR:Fc may be used to treat hepatitis C, including chronic hepatitis C, in patients who are concurrently treated with interferon α. High expression of TNFα in the liver interferes with the action of IFNα, thus interfering with the patient's response to IFNα treatment (Hong et al., FASEB J 15:1595-97 (2001)). Treatments that may be administered concurrently with a TNFα inhibitor to treat hepatitis C include pegylated IFNα, ribavirin, or a combination of ribavirin and interferon α or pegylated interferon α. Interferon α moieties suitable for concurrent use with a TNFα inhibitor include IFNα-2a (such as ROFERON®; Hoffmann-LaRoche), pegylated IFNα-2a (such as PEGASYS®; Hoffmann-LaRoche), pegylated IFNα-2a or −2b as described in US20020127203A1 or the pegylated-IFNα conjugates described in WO 9964016. Hepatitis C may be treated also by concurrent administration of interferon α and another TNFα inhibitor other than TNFR:Fc, such as lenercept, onercept, infliximab, adalimumab, afelimomab, CDP571 or another TNFα inhibitor.

A TNFα inhibitor can be administered to cancer patients to reduce the undesired side effects associated with long-term interferon administration, which can include fatigue, fever, neutropenia, rash, headache, digestive disorders, liver enzyme imbalances and so on. For example, interferon γ (IFNγ) has been shown to be active in ovarian cancer, thus a patient with ovarian cancer can be treated by concurrently administering IFNγ and a TNFα inhibitor, such as TNFR:Fc, or an antibody specific for TNFα. Similarly, IFNα is often used to treat melanoma, chronic myelogenous leukemia, basal cell carcinoma, hairy cell leukemia, bladder cancer, hemangiomas of infancy and childhood, multiple myeloma, Kaposi's sarcoma, mycosis fungoides, non-Hodgkin's lymphoma and renal cell carcinoma and can be administered concurrently with a TNFα inhibitor, such as TNFR:Fc, to reduce interferon-induced side effects.

TNFR:Fc or TNFR:Fc combined with the cytokine IFNγ-1b (such as ACTIMMUNE®; InterMune Pharmaceuticals) may be used for treating cystic fibrosis or fibrotic lung diseases, such as idiopathic pulmonary fibrosis, radiation-induced pulmonary fibrosis and bleomycin-induced pulmonary fibrosis. In addition, this combination is useful for treating other diseases characterized by organ fibrosis, including systemic sclerosis (also called “scleroderma”), which often involves fibrosis of the liver. For treating cystic fibrosis, TNFR:Fc and IFNγ-1b may be combined with PULMOZYME® or TOBI® or other treatments for cystic fibrosis.

TNFR:Fc alone or in combination with IFNγ-1b may be administered together with other treatments presently used for treating fibrotic lung disease. Such additional treatments include glucocorticoids, azathioprine, cyclophosphamide, penicillamine, colchisine, supplemental oxygen and so forth. Patients with fibrotic lung disease, such as IPF, often present with nonproductive cough, progressive dyspnea, and show a restrictive ventilatory pattern in pulmonary function tests. Chest radiographs reveal fibrotic accumulations in the patient's lungs. When treating fibrotic lung disease in accord with the disclosed methods, sufficiency of treatment may be detected by observing a decrease in the patient's coughing (when cough is present), or by using standard lung function tests to detect improvements in total lung capacity, vital capacity, residual lung volume or by administering a arterial blood gas determination measuring desaturation under exercising conditions, and showing that the patient's lung function has improved according to one or more of these measures. In addition, patient improvement may be determined through chest radiography results showing that the progression of fibrosis in the patient's lungs has become arrested or reduced.

In addition, TNF inhibitors (including soluble TNFRs or antibodies against TNFα or TNFR) are useful for treating organ fibrosis when administered in combination with relaxin, a hormone that down-regulates collagen production thus inhibiting fibrosis, or when given in combination with agents that block the fibrogenic activity of TGF-β. Combination therapies using TNFR:Fc and recombinant human relaxin are useful, for example, for treating systemic sclerosis or fibrotic lung diseases, including cystic fibrosis, idiopathic pulmonary fibrosis, radiation-induced pulmonary fibrosis and bleomycin-induced pulmonary fibrosis.

Other embodiments provide methods for using the disclosed TNFα inhibitors, compositions or combination therapies to treat a variety of rheumatic disorders. These include: adult and juvenile rheumatoid arthritis; systemic lupus erythematosus; gout; osteoarthritis; polymyalgia rheumatica; seronegative spondylarthropathies, including ankylosing spondylitis; and Reiter's disease (reactive arthritis). The subject TNFα inhibitors, compositions and combination therapies are used also to treat psoriatic arthritis and chronic Lyme arthritis. Also treatable with these compounds, compositions and combination therapies are Still's disease and uveitis associated with rheumatoid arthritis. In addition, the compounds, compositions and combination therapies of the invention are used in treating disorders resulting in inflammation of the voluntary muscle, including dermatomyositis and polymyositis. Moreover, the compounds, compositions ant combinations disclosed herein are useful for treating sporadic inclusion body myositis, as TNFα may play a significant role in the progression of this muscle disease. In addition, the compounds, compositions and combinations disclosed herein are used to treat multicentric reticulohistiocytosis, a disease in which joint destruction and papular nodules of the face and hands are associated with excess production of proinflammatory cytokines by multinucleated giant cells.

The TNFα inhibitors, compositions and combination therapies of the invention may be used to inhibit hypertrophic scarring, a phenomenon believed to result in part from excessive TNFα secretion. TNF inhibitors may be administered alone or concurrently with other agents that inhibit hypertrophic scarring, such as inhibitors of TGF-α.

Cervicogenic headache is a common form of headache arising from dysfunction in the neck area, and which is associated with elevated levels of TNFα, which are believed to mediate an inflammatory condition that contributes to the patient's discomfort (Martelletti, Clin Exp Rheumatol 18(2 Suppl 19):S33-8 (March-April, 2000)). Cervicogenic headache may be treated by administering an inhibitor of TNFα as disclosed herein, thereby reducing the inflammatory response and associated headache pain.

The TNFα inhibitors, compositions and combination therapies of the invention are useful for treating primary amyloidosis. In addition, the secondary amyloidosis that is characteristic of various conditions also are treatable with TNFα inhibitors such as TNFR:Fc, and the compositions and combination therapies described herein. Such conditions include: Alzheimer's disease, secondary reactive amyloidosis; Down's syndrome; and dialysis-associated amyloidosis. Also treatable with the compounds, compositions and combination therapies of the invention are inherited periodic fever syndromes, including familial Mediterranean fever, hyperimmunoglobulin D and periodic fever syndrome and TNF-receptor associated periodic syndromes (TRAPS).

Disorders associated with transplantation also are treatable with the disclosed TNFα inhibitors, compositions or combination therapies, such as graft-versus-host disease, and other complications resulting from solid organ transplantation, including transplantation of heart, liver, lung, skin, kidney or other organs. Such inhibitors may be administered, for example, to prevent or inhibit the development of bronchiolitis obliterans, such as bronchiolitis obliterans after lung transplantation and bronchiolitis obliterans organizing pneumonia. Patients undergoing autologous hematopoietic stem cell transplantation in the form of peripheral blood stem cell transplantation may develop “engraftment syndrome,” or “ES,” which is an adverse and generally self-limited response that occurs about the time of hematopoietic engraftment and which can result in pulmonary deterioration. ES may be treated with inhibitors of either IL-8 or TNFα (such as TNFR:Fc), or with a combination of inhibitors against both of these cytokines. The disclosed TNFα inhibitors also are useful for treating or preventing graft failure, such as bone marrow graft rejection or failure of the recipient's body to accept other types of grafts, such as corneal transplants, or such as liver or other solid organ transplants, in which graft rejection is often accompanied by elevated levels of TNFα and IL-10. Graft rejection may be treated with a combination of a TNFα inhibitor and an IL-10 inhibitor.

Ocular disorders also are treatable with the disclosed TNFα inhibitors, compositions or combination therapies, including rhegmatogenous retinal detachment, and inflammatory eye disease, and inflammatory eye disease associated with smoking as well as macular degeneration associated with smoking or associated with aging.

TNFα inhibitors such as TNFR:Fc and the disclosed compositions and combination therapies also are useful for treating disorders that affect the female reproductive system. Examples include, but are not limited to, multiple implant failure/infertility; fetal loss syndrome or IV embryo loss (spontaneous abortion); preeclamptic pregnancies or eclampsia; and endometriosis.

In addition, the disclosed TNFα inhibitors, compositions and combination therapies are useful for treating obesity, including treatment to bring about a decrease in leptin formation, or weight gain associated with the use of anti-depressant medications. Also, the compounds, compositions and combination therapies of the invention are used to treat neurogenic pain, sciatica, symptoms of aging, severe drug reactions (for example, II-2 toxicity or bleomycin-induced pneumopathy and fibrosis), or to suppress the inflammatory response prior, during or after the transfusion of allogeneic red blood cells in cardiac or other surgery, or in treating a traumatic injury to a limb or joint, such as traumatic knee injury. Various other medical disorders treatable with the disclosed TNFα inhibitors, compositions and combination therapies include; multiple sclerosis; Behcet's syndrome; Sjogren's syndrome; autoimmune hemolytic anemia; beta thalassemia; amyotrophic lateral sclerosis (Lou Gehrig's Disease); Parkinson's disease; and tenosynovitis of unknown cause, as well as various autoimmune disorders or diseases associated with hereditary deficiencies.

The disclosed TNFα inhibitors, compositions and combination therapies furthermore are useful for treating acute polyneuropathy; anorexia nervosa; Bell's palsy; chronic fatigue syndrome; transmissible dementia, including Creutzfeld-Jacob disease; demyclinating neuropathy; Guillain-Barre syndrome; vertebral disc disease; Gulf war syndrome; myasthenia gravis; silent cerebral ischemia; sleep disorders, including narcolepsy and sleep apnea; chronic neuronal degeneration; and stroke, including cerebral ischemic diseases.

Disorders involving the skin or mucous membranes also are treatable using the disclosed TNFα inhibitors, compositions or combination therapies. Such disorders include acantholytic diseases, including Darier's disease, keratosis follicularis and pemphigus vulgaris. Also treatable with the subject TNFα inhibitors, compositions and combination therapies are acne; acne rosacea; alopecia areata; aphthous stomatitis; bullous pemphigoid; burns; dermatitis herpetiformis; eczema; erythema, including erythema multiforme and erythema multiforme bullosum (Stevens-Johnson syndrome); inflammatory skin disease; lichen planus; linear IgA bullous disease (chronic bullous dermatosis of childhood); loss of skin elasticity; mucosal surface ulcers; neutrophilic dermatitis (Sweet's syndrome); pityriasis rubra pilaris; psoriasis; pyoderma gangrenosum; and toxic epidermal necrolysis.

In another embodiment, the disclosed TNFα inhibitors are used to treat and prevent the recurrence of lipodermatosclerosis and chronic venous ulcers, which most often are located on the legs. Studies have shown that TNFα may contribute to the pathogenesis of lipodermatosclerosis and chronic venous ulcers by activation of matrix metalloproteinase 2 (MMP2), and by inducing the production of TGFα and other cytokines. Oxpentifylline and pentoxifylline have been shown to be effective in this setting. The disclosed TNFα inhibitors, including TNFR:Fc or antibodies to TNFα, may be used to treat chronic venous ulcers either alone or in combination with one or more of oxpentifylline, pentoxifylline, GM-CSF, leptin, PDGF, bFGF, EGF, TGF and/or IGF. These treatments will accelerate healing and prevent recurrences. Administration may be systemic or local. For local administration, the TNFα inhibitor is applied topically in an ointment, lotion, gel or cream, or is injected perilesionally directly into or within about ten centimeters of the ulcer.

Transfection of lymphocytes with non-viral vectors can lead to apoptosis of the target cells through a TNFα and CD95-mediated pathway (see, for example, Ebert et al., Cytokines, Cell & Mol Ther 5:165-73 (1999)). Soluble TNFRs, such as TNFR:Fc, may be used alone or in combination with a CD95 inhibitor, such as an antibody against CD95, to inhibit this apoptosis. This treatment will augment gene transfer to lymphocytes when non-viral vectors are used, particularly when liposome-mediated or receptor-mediated gene transfer methods are used. Such treatment will improve the incorporation of the exogenous gene into the target cells.

Any of the disclosed TNF inhibitors or combination treatments disclosed herein also may be used to treat familial combined hyperlipidemia (FCH). FHC is a genetic dyslipidemia characterized by premature coronary heart disease. FCH patients are genetically defective in their TNFR II gene, have low levels of sTNFR II levels in their bodies and appear to be hyperresponsive to the deleterious effects of endogenous TNFα (van Greevenbroek et al., Atherosclerosis 153:1-8 (2000)). Coronary heart disease, insulin resistance and obesity associated with FCH can be ameliorated or prevented by administering to FCH patients any one of the TNF inhibitors disclosed herein, such as TNFR:Fc or an antibody against TNFα. In addition, TNFα antagonist treatment for FCH may be administered concurrently with reduction of dietary fat and cholesterol and/or with one or more of the other drugs used to treat this condition, including bile acid-sequestering resins (cholestyramine and colestipol), nicotinic acid, niacin, a cholesterol-lowering drug, such as gemfibrozil or probucol, or one of the cholesterol-lowering “statin” or HMG-CoA reductase inhibitors, such as lovastatin or pravastatin.

In another aspect of the invention, TNF inhibitors are used to treat patients who have elevated serum levels of C-reactive protein (CRP) and who thus are at risk for heart attack even when their cholesterol may be low (Ridker et al., New Eng J Med 344:1959-65 (2001)).

In yet another embodiment of the invention, the TNF inhibitors disclosed herein are used to treat autism spectrum disorder and other pervasive developmental disorders. It has been shown that proinflammatory cytokines, including TNFα and IL-1 are overproduced in a subset of autistic patients, indicating that these patients had excessive innate immune responses and/or aberrant production of regulatory cytokines for T cell responses. Thus, provided herein are methods for treating autism spectrum disorder by administering a TNF inhibitor such as TNFR:Fc or another of the TNF inhibitors described herein.

Various other medicaments used to treat the diseases described herein may also be administered concurrently with compositions comprising TNFα inhibitors, such as TNFR:Fc. Such medicaments include: NSAIDs; DMARDs; analgesics; topical steroids; systemic steroids (e.g., prednisone); cytokines; antagonists of inflammatory cytokines; antibodies against T cell surface proteins; oral retinoids; salicylic acid; and hydroxyurea. Suitable analgesics for such combinations include: acetaminophen, codeine, propoxyphene napsylate, oxycodone hydrochloride, hydrocodone bitartrate and tramadol. DMARDs suitable for such combinations include: azathioprine, cyclophosphamide, cyclosporine, hydroxychloroquine sulfate, methotrexate, leflunomide, minocycline, penicillamine, sulfasalazine, oral gold, gold sodium thiomalate and aurothioglucose. In addition, the TNFR:Fc or other TNFR mimic may be administered in combination with antimalarials or colchicine. NSAIDs suitable for the subject combination treatments include: salicylic acid (aspirin) and salicylate derivatives; ibuprofen; indomethacin; celecoxib (CELEBREX®); rofecoxib (VIOXX®); valdecoxib (BEXTRA®) ketorolac; nambumetone; piroxicam; naproxen; oxaprozin; sulindac; ketoprofen; diclofenac; and other COX-1 and COX-2 inhibitors, propionic acid derivatives, acetic acid derivatives, fumaric acid derivatives, carboxylic acid derivatives, butyric acid derivatives, oxicams, pyrazoles and pyrazolones, including newly developed anti-inflammatories.

In addition to human patients, inhibitors of TNFα are useful in the treatment of autoimmune and inflammatory conditions in non-human animals, such as pets (dogs, cats, birds, primates, etc.), domestic farm animals (horses cattle, sheep, pigs, birds, etc.), or any animal that suffers from a TNFα-mediated inflammatory or arthritic condition comparable to one of the conditions described herein. In such instances, an appropriate dose may be determined according to the animal's body weight. For example, a dose of 0.2-1 mg/kg may be used. Alternatively, the dose is determined according to the animal's surface area, an exemplary dose ranging from 0.1-20 mg/m of body surface area, or more preferably, from 5-12 mg/m² of body surface area. For small animals, such as dogs or cats, a suitable dose is 0.4 mg/kg of body weight. In another embodiment, TNFR:Fc (preferably constructed from genes derived from the same species as the patient), or another soluble TNFR mimic, is administered by injection or other suitable route one or more times per week until the animal's condition is improved, or it may be administered indefinitely.

What is claimed is:

1. A method of preventing or reducing resistance to imatinib in a chronic myelogenous leukemia patient comprising administering TNFR:Fc to a chronic myelogenous leukemia patient, wherein said patient is treated concurrently with imatinib.

2. The method of claim 1, wherein the TNFR:Fc is administered one or more times per week.

3. The method of claim 2, wherein TNFR:Fc is administered by subcutaneous injection.

4. The method of claim 3, wherein the patient is an adult and the amount of TNFR:Fc injected is 5-12 mg/m² of body surface area, or 25 mg or 50 mg per dose.

5. The method of claim 1, wherein the patient is a pediatric patient and the TNFα inhibitor is TNFR:Fc, and further wherein the TNFR:Fc is administered by subcutaneous injection one or more times per week at a dose of 0.4 mg/kg of body weight, up to a maximum of 25 mg per dose.

6. A method of preventing or reducing resistance to UCN-01 comprising administering TNFR:Fc to a patient having gastrointestinal cancer, wherein said patient is treated concurrently with UCN-01.

7. The method of claim 6, wherein the TNFR:Fc is administered one or more times per week.

8. The method of claim 7, wherein TNFR:Fc is administered by subcutaneous injection.

9. The method of claim 8, wherein the patient is an adult and the amount of TNFR:Fc injected is 5-12 mg/m of body surface area, or 25 mg or 50 mg per dose.

10. The method of claim 6, wherein the patient is a pediatric patient and the TNFR:Fc is administered by subcutaneous injection one or more times per week at a dose of 0.4 mg/kg of body weight, up to a maximum of 25 mg per dose.
